(19) United States
(12) Patent Application Publication
(10) Pub. No.: US 2009/0226490 A1
(43) Pub. Date: Sep. 10, 2009

(54) Use of Whole Bacterial Cells (Actinomycetales) for Maternal Administration to Modulate Offspring Immune Response

(76) Inventors: John Lawson Stanford, Kent (GB); Cynthia Ann Stanford, Kent (GB); Graham McIntyre, Kent (GB); Oscar Adelmo Bottasso, Provincia de Santa Fe (AR)

Correspondence Address: STEPTOE & JOHNSON LLP, 1330 CONNECTICUT AVENUE, N.W., WASHINGTON, DC, 20036, US

(21) Appl. No.: 12/086,422

(22) PCT Filed: Dec. 19, 2006
(86) PCT No.: PCT/GB2006/004793

§ 371 Date: Oct. 28, 2008

(30) Foreign Application Priority Data
Dec. 21, 2005 (GB) 0526031.0

(51) Int. Cl. A61K 39/02

(52) U.S. Cl. 424/234.1

(57) ABSTRACT

Use of a composition comprising a whole cell of a bacterium from a genus of aerobic organisms in the order of Actinomycetales in the manufacture of a medicament for modulating the immune response of a recipient wherein said composition is administered to a subject and said recipient is exposed to a bodily of said subject whilst said recipient is an infant. Preferably the medicament is used for the prevention and/or treatment of one or more of: an infection, an allergy, an autoimmune-based disease and a neoplasm in said recipient.

### FIELD OF INVENTION

The present invention relates to maternal immunomodulation. In particular the present invention relates to the administration of a whole cell of a bacterium from a genus of aerobic organisms in the order Actinomycetales to a subject in order to immunomodulate a recipient. In addition, the present invention relates to methods of using a whole cell of a bacterium from a genus of aerobic organisms in the order Actinomycetales to provide a subject with the ability to immunomodulate a recipient.

### BACKGROUND TO THE INVENTION

The immune system of an infant is immature and thus an infant is more susceptible to, for example, infectious disease. Furthermore, it is difficult to induce protective immunity directly, such as by vaccination, during the neonatal period.

Davila HO et al. (Am. J. Trop. Hyg., 50(4), 1994, pp 506-511) and (Am. J. Trop. Hyg., 54(6), 1996, pp 660-664) disclose that infection with Trypanosoma cruzi and/or interferon-gamma during pregnancy gives protection to T. cruzi in the off-spring.

Lundin BS, et al. (Scand. J. Immunol. 1999; 50, 651-656), disclose that monoclonal antibodies directed against one bacterial component given in the neonatal period can affect the humoral immune response to bacteria containing that component for two generations.

It has been suggested that individual components of bacterial cells could be used to elicit an adjuvant effect and an immune modulator composition comprising a whole cell of a bacterium from the genera Rhodococcus, Gordona, Nocardia, Dietzia, Tsukamurella and Nocardioides has been taught (see WO204/022093 and UK patent application number 0404102.6 both incorporated herein by reference). However, to date it has neither been taught nor suggested that administration of a whole cell of a bacterium from a genus of aerobic organisms in the order of Actinomycetales to a subject may modulate the immune system of an infant by, for example, exposing the immune system of the infant to a bodily fluid of the subject.

### SUMMARY OF THE INVENTION

A seminal finding of the present invention is that a subject, when administered a whole cell of a bacterium from a genus of aerobic organisms in the order of Actinomycetales, has the ability to modulate, preferably enhance, the immune system of a recipient, following the exposure of the recipient to a bodily fluid of the subject whilst the recipient is an infant.

### DETAILED ASPECTS OF THE INVENTION

In one aspect, the present invention relates to the use of a composition comprising a whole cell of a bacterium from a genus of aerobic organisms in the order of Actinomycetales (and optionally in conjunction with a pharmaceutically acceptable carrier, diluent and/or excipient) in the manufacture of a medicament for modulating, preferably enhancing, the immune response of a recipient wherein said composition is administered to a subject and said recipient is exposed to a bodily fluid of said subject whilst said recipient is an infant.

In another aspect, the present invention relates to the use of a composition comprising a whole cell of a bacterium from a genus of aerobic organisms in the order of Actinomycetales (and optionally in conjunction with a pharmaceutically acceptable carrier, diluent and/or excipient) in the manufacture of a medicament for the treatment or prevention (preferably prevention) of one or more diseases or disorders selected from the group consisting of: an infection, an allergy, an autoimmune based disease and a neoplasm in a recipient wherein said composition is administered to a subject and said recipient is exposed to a bodily fluid of said subject whilst said recipient is an infant.

In another aspect, the present invention relates to the use of a composition comprising a whole cell of a bacterium from a genus of aerobic organisms in the order of Actinomycetales (and optionally in conjunction with a pharmaceutically acceptable carrier, diluent and/or excipient) in the manufacture of a medicament for the treatment or prevention (preferably prevention) of one or more of diseases or disorders selected from the group consisting of: an infection (for example a bacterial infection, a viral infection, such as HIV, PMWS and PDNS, an infection caused by papilloma viruses, including equine sarcoid, genital warts and dysplasia of the uterine cervix that precedes carcinoma of the cervix, or a parasitic infection, such as malaria, and visceral infections, such as leishmaniasis for example); an immunological abnormality accompanying an infection; an autoimmune disease (e.g. a vascular disorder, such as obliterative vascular disorder, and the immunological aspects underlying myointimal hyperplasia and/or atheroma formation (otherwise known as arteriosclerosis), diabetes, arthritis and graft rejection); stress (for example, major trauma stress, psychosocial stress and chronic stress); an allergy (for example asthma including allergic asthma, hayfever, allergic dermatitis (eczema), allergies to plant contact or ingestion, stings—such as nettle and insect stings, and allergies to insect bites—such as midges, for instance Culicoides (which causes Sweet Itch in horses), and fleas); heaves; COPD; autism; SIPH (such as EIPH); cancer (for example leukaemia, rare solid tumours of childhood, melanoma, carcinoma, sarcoma or adenocarcinoma); an immune system imbalance (e.g. an immune system imbalance in children); and post-operative stress and infection in a recipient wherein said composition is administered to a subject and said recipient is exposed to a bodily fluid of said subject whilst said recipient is an infant.

Suitably, the medicament may be for the treatment or prevention (preferably prevention) of an infection in a recipient. Suitably, the infection may be selected from the group consisting of: a bacterial infection, a viral infection (preferably HIV) or a parasitic infection (preferably malaria and/or a visceral infection (such as leishmaniasis). Preferably the infection is HIV, malaria and/or a visceral infection.

Suitably, the medicament may be for the treatment or prevention (preferably prevention) of an autoimmune disease in a recipient. Suitably the autoimmune disease may be selected from the group consisting of: diabetes, a vascular disorder, such as an obliterative vascular disorder and the immunological aspects underlying myointimal hyperplasia and/or atheroma formation (otherwise known as arteriosclerosis), arthritis and graft rejection. Preferably the autoimmune disease may be diabetes and/or arthritis. Suitably, the medicament may be for the treatment or prevention (preferably prevention) of an allergy. Suitably the allergy may be selected from the group consisting of: asthma (including allergic asthma), hayfever, allergic dermatitis (eczema), allergies to plant contact or ingestion, stings—such as nettle and insect stings, and allergies to insect bites—such as midges, for instance Culicoides (which causes Sweet Itch in horses) and fleas. Preferably the allergy is a food allergy (e.g. a nut allergy) and/or an allergy to an insect bite (e.g. a flea allergy).

Suitably, the medicament may be for the treatment or prevention (preferably prevention) of a cancer. Suitably the cancer may be selected from the group consisting of: leukaemia, rare solid tumour of childhood, melanoma, carcinoma, adenocarcinoma and sarcoma. Preferably the cancer is an adenocarcinoma and/or a sarcoma.

Suitably, the medicament may be for the treatment or prevention (preferably prevention) of an immune system imbalance. Suitably, the immune system imbalance may be in child.

In a further aspect, the present invention relates to a method of providing a subject with the ability to modulate, preferably enhance, the immune response of a recipient comprising administering a composition comprising an effective amount of a whole cell of a bacterium from a genus of aerobic organisms in the order of Actinomycetales (and optionally a pharmaceutically acceptable carrier, diluent and/or excipient) to a subject and exposing the recipient to a bodily fluid of said subject whilst said recipient is an infant.

In one aspect, the present invention relates to a method of modulating the immune response of a recipient comprising administering a composition comprising an effective amount of a composition comprising whole cells of a bacterium from a genus of aerobic organisms in the order of Actinomycetales to a subject and exposing the recipient to a bodily fluid of said subject whilst said recipient is an infant.

In another aspect, the present invention relates to a method of modulating (preferably enhancing) the immune response of a recipient comprising administering a composition comprising an effective amount of a whole cell of a bacterium from a genus in the order of Actinomycetales (and optionally a pharmaceutically acceptable carrier, diluent and/or excipient) to a subject prior to and/or during pregnancy with said recipient, such that the immunity of said recipient is modulated (preferably enhanced).

In a further aspect, the present invention relates to a method of modulating (preferably enhancing) the immune response of a recipient comprising administering a composition comprising an effective amount of a whole cell of a bacterium from a genus in the order of Actinomycetales (and optionally a pharmaceutically acceptable carrier, diluent and/or excipient) to a subject prior to lactation and feeding the recipient with the milk whilst said recipient is an infant, such that the immunity of said recipient is modulated (preferably enhanced).

In another aspect, the present invention relates to a method for maternally derived protection of a recipient from one or more diseases or disorders selected from the group consisting of: an infection, an allergy, an autoimmune based disease and a neoplasm comprising administering a composition comprising a whole cell of a bacterium from a genus of aerobic organisms in the order of Actinomycetales (and optionally in conjunction with a pharmaceutically acceptable carrier, diluent and/or excipient) to a subject and exposing said recipient to a bodily fluid of said subject whilst said recipient is an infant.

In yet another aspect, the present invention relates to a method for maternally derived protection of a recipient from one or more of diseases or disorders selected from the group consisting of: an infection (for example a bacterial infection, a viral infection, such as HIV, PMWS and PDNS, an infection caused by papilloma viruses, including equine sarcoid, genital warts and dysplasia of the uterine cervix that precedes carcinoma of the cervix, or a parasitic infection, such as malaria, and visceral infections); an immunological abnormality accompanying an infection; an autoimmune disease (e.g. a vascular disorder, such as obliterative vascular disorder, and the immunological aspects underlying myointimal hyperplasia and/or atheroma formation (otherwise known as arteriosclerosis), diabetes, arthritis and graft rejection); stress (for example, major trauma stress, psychosocial stress and chronic stress); an allergy (for example asthma including allergic asthma, hayfever, allergic dermatitis (eczema), allergies to plant contact or ingestion, stings—such as nettle and insect stings, and allergies to insect bites—such as midges, for instance Culicoides (which causes Sweet Itch in horses) and fleas); heaves; COPD; autism; SIPH (such as EIPH); cancer (for example leukaemia, rare solid tumours of childhood, melanoma, carcinoma, sarcoma or adenocarcinoma); an immune system imbalance (e.g. an immune system imbalance in children); and post-operative stress (including stress associated with the administration of anesthetics for instance) and post-operative infection.

The terms “exposed”, “exposing”, and “exposure” as used herein means the act of bringing the recipient as an infant into contact with a bodily fluid of the subject or a part thereof. Preferably the exposure is oral (i.e. the recipient as an infant ingests the bodily fluid) and/or transplacental (i.e. the exposure occurs across the placenta).

When the exposure occurs by a recipient as an infant ingesting the bodily fluid of a subject, preferably the bodily fluid is the milk of a lactating subject.

When the exposure occurs transplancental the bodily fluid of the subject (i.e. the parent) enters the placenta and one or more components thereof may transfer across the placenta. In the present application this may be considered as the recipient being exposed to the bodily fluid of the subject, even though the bodily fluid or part thereof may not come directly into contact with the recipient.

### Preferable Aspects

The composition may be an immune modulator composition and/or a pharmaceutical composition. The pharmaceutical composition may additionally comprise a pharmaceutically acceptable carrier, diluent and/or excipient.

The present invention further provides the use of an immune modulator composition or a pharmaceutical composition comprising a whole cell of a bacterium from a genus of aerobic organisms in the order of Actinomycetales, in the manufacture of a medicament for the treatment or prevention (preferably prevention) of a bacterial infection in a recipient wherein said composition is administered to a subject and said recipient is exposed to a bodily fluid of said subject whilst said recipient is an infant.

The present invention further provides the use of an immune modulator composition or a pharmaceutical composition comprising a whole cell of a bacterium from a genus of aerobic organisms in the order of Actinomycetales, in the manufacture of a medicament for the prevention or treatment (preferably prevention) of a parasitic infection, such as, for example, malaria, in a recipient wherein said composition is administered to a subject and said recipient is exposed to a bodily fluid of said subject whilst said recipient is an infant.

The present invention further provides the use of an immune modulator composition or a pharmaceutical composition comprising a whole cell of a bacterium from a genus of aerobic organisms in the order of Actinomycetales, in the manufacture of a medicament for the treatment or prevention (preferably prevention) of a viral infection, such as HIV, in a recipient wherein said composition is administered to a subject and said recipient is exposed to a bodily fluid of said subject whilst said recipient is an infant.

In this way, protection against a mother's HIV infection may be provided to a recipient.

The present invention further provides the use of an immune modulator composition or a pharmaceutical composition comprising a whole cell of a bacterium from a genus of aerobic organisms in the order of Actinomycetales, in the manufacture of a medicament for the treatment or prevention (preferably prevention) of an allergy (for example asthma (including allergic asthma), hayfever, allergic dermatitis (eczema), allergies to plant contact or ingestion, stings—such as nettle and insect stings, and allergies to insect bites—such as midges for instance Culicoides (which causes Sweet Itch in horses)) in a recipient wherein said composition is administered to a subject and said recipient is exposed to a bodily fluid of said subject whilst said recipient is an infant.

The present invention further provides the use of an immune modulator composition or a pharmaceutical composition comprising a whole cell of a bacterium from a genus of aerobic organisms in the order of Actinomycetales, in the manufacture of a medicament for the treatment or prevention (preferably prevention) of one or more of heaves and/or COPD in an infant wherein said composition is administered to a subject and said recipient is exposed to a bodily fluid of said subject whilst said recipient is an infant. Preferably the recipient is a horse. Suitably, the medicament may be a veterinary medicament.

The present invention further provides the use of an immune modulator composition or a pharmaceutical composition comprising a whole cell of a bacterium from a genus of aerobic organisms in the order of Actinomycetales, in the manufacture of a medicament for the treatment or prevention (preferably prevention) of one or more of PMWS and/or PDNS in a recipient wherein said composition is administered to a subject and said recipient is exposed to a bodily fluid of said subject whilst said recipient is an infant.

The present invention further provides the use of an immune modulator composition or a pharmaceutical composition comprising a whole cell of a bacterium from a genus of aerobic organisms in the order of Actinomycetales, in the manufacture of a medicament to for the treatment or prevention (preferably prevention) of a cancer in a recipient wherein said composition is administered to a subject and said recipient is exposed to a bodily fluid of said subject whilst said recipient is an infant. Suitably the cancer may be a leukaemia, a rare solid tumour, a melanoma and/or an adenocarcinoma in a recipient.

The present invention further provides the use of an immune modulator composition or a pharmaceutical composition comprising a whole cell of a from a genus of aerobic organisms in the order of Actinomycetales, in the manufacture of a medicament for the treatment or prevention (preferably prevention) of an immune system imbalance in a recipient wherein said composition is administered to a subject and said recipient is exposed to a bodily fluid of said subject whilst said recipient is an infant.

The present invention further provides the use of an immune modulator composition or a pharmaceutical composition comprising a whole cell of a from a genus of aerobic organisms in the order of Actinomycetales, in the manufacture of a medicament for enhancing the immune system in a recipient wherein said composition is administered to a subject and said recipient is exposed to a bodily fluid of said subject whilst said recipient is an infant.

When for example the immune system of a recipient is enhanced this may lead to an enhancement of growth or an increase in the efficiency of feed utilisation in the recipient as an infant pre- and post-birth. Growth enhancement and/or increased efficiency in feed utilisation may be of particular importance in livestock, such as pig and/or cattle, and may be particularly important in piglets and/or calves.

Typically, the immune modulator composition or pharmaceutical composition according to this aspect of the present invention may be an immune enhancer.

Advantageously, the immune modulator composition or pharmaceutical composition of the present invention may be used to replace antibiotics that are currently used to promote the growth of livestock.

Suitably, the immune modulator composition of the present invention may be used either alone or in combination with other treatments.

The present invention further provides the use of an immune modulator composition or a pharmaceutical composition comprising a whole cell of a bacterium from a genus of aerobic organisms in the order of Actinomycetales, in the manufacture of a medicament for the treatment or prevention (preferably prevention) of an adverse reaction to childhood vaccines—such as whooping cough vaccinations and the current MMR vaccinations—and/or consequences thereof in a recipient wherein said composition is administered to a subject and said recipient is exposed to a bodily fluid of said subject whilst said recipient is an infant.

The term “adverse reaction”, as used herein, means a local or generalised disadvantageous response caused by or primed by the vaccine or the administration thereof, which typically occurs within a short time-frame but which can be delayed (for example by 6-months). An “adverse reaction” may include death of the child. The adverse reaction may be caused as a consequence of a separate event, the response to which has been negatively primed by the vaccine or the administration thereof. In one embodiment, an adverse reaction to vaccination may be induced by adjuvants present in the vaccine which cause a disadvantageous presentation of cellular immunity, such as the production of both Th1 and Th2 simultaneously which leads to tissue damage.

The present invention further provides a method of providing a subject with the ability to treat or prevent (preferably prevent) one or more bacterial infections in a recipient comprising administering an effective amount of a whole cell of a bacterium from a genus of aerobic organisms in the order of Actinomycetales to a subject and exposing the recipient to a bodily fluid of said subject whilst said recipient is an infant.

The present invention further provides a method of providing a subject with the ability to treat or prevent (preferably prevent) one or more parasitic infections, such as, for example, malaria in a recipient, comprising administering an effective amount of a whole cell of a bacterium from a genus of aerobic organisms in the order of Actinomycetales to a subject and exposing the recipient to a bodily fluid of said subject whilst said recipient is an infant.

The present invention further provides a method of providing a subject with the ability to treat or prevent (preferably prevent) a viral infection, such as HIV, in a recipient comprising administering an effective amount of a whole cell of a bacterium from a genus of aerobic organisms in the order of Actinomycetales to a subject and exposing the recipient to a bodily fluid of said subject whilst said recipient is an infant.

The present invention further provides a method of providing a subject with the ability to treat or prevent (preferably prevent) one or more allergies (for example asthma (including allergic asthma), hayfever, allergic dermatitis (eczema), allergies to plant contact or ingestion, stings—such as nettle and insect stings, and allergies to insect bites—such as midges for instance Culicoides (which causes Sweet Itch in horses) or fleas) in a recipient, comprising administering an effective amount of a whole cell of a bacterium from a genus of aerobic organisms in the order of Actinomycetales to a subject and exposing the recipient to a bodily fluid of said subject whilst said recipient is an infant.

Preferably, the method is used to treat or prevent (preferably prevent) asthma including allergic asthma, and allergies to insect bites—such as midges for instance Culicoides (which causes Sweet Itch in horses) or fleas.

The present invention further provides a method of providing a subject (e.g. a horse) with the ability to prevent one or more of heaves and/or COPD in a recipient (e.g. another horse), comprising administering an effective amount of a whole cell of a bacterium from a genus of aerobic organisms in the order of Actinomycetales to a subject and exposing the recipient to a bodily fluid of said subject whilst said recipient is an infant (e.g. a foal).

The present invention further provides a method of providing a subject (e.g. a pig) with the ability to prevent one or more of PMWS and/or PDNS in a recipient (e.g. another pig), comprising administering an effective amount of a whole cell of a bacterium from a genus of aerobic organisms in the order of Actinomycetales to a subject and exposing the recipient to a bodily fluid of said subject whilst said recipient is an infant (e.g. a piglet).

The present invention further provides a method of providing a subject with the ability to treat or prevent (preferably prevent) one or more cancer (such as leukaemias, adenocarcinomas, melanomas or rare solid cancers) in a recipient, comprising administering an effective amount of a whole cell of a bacterium from a genus of aerobic organisms in the order of Actinomycetales to a subject and exposing the recipient to a bodily fluid of said subject whilst said recipient is an infant.

The present invention further provides a method of providing a subject with the ability to treat or prevent (preferably prevent) an immune system imbalance in an infant, comprising administering an effective amount of a whole cell of a bacterium from a genus of aerobic organisms in the order of Actinomycetales to a subject and exposing the infant to a bodily fluid of said subject whilst said recipient is an infant.

The present invention further provides a method of providing a subject with the ability to enhance the immune system in a recipient, comprising administering an effective amount of a whole cell of a bacterium from a genus of aerobic organisms in the order of Actinomycetales to a subject and exposing the recipient to a bodily fluid of said subject whilst said recipient is an infant.

Such immune system enhancement may result in for example, enhancement of growth or an increase in the efficiency of feed utilisation in said recipient pre- or post-birth.

Advantageously, the method of the present invention may be used to replace antibiotics that are currently used to promote the growth of livestock.

Suitably, the method of the present invention may be used either alone or in combination with other treatments.

The term “maternally derived protection” and/or “maternal immunomodulation” as used herein means that the recipient (as an infant) gains protection through “a mother” whether the mother is the recipient's actual mother, a surrogate mother or a wet nurse (i.e. a subject employed to suckle another subject's infant). Suitably, the term “a mother” used herein refers to the actual mother of the recipient or to a subject that has at least recently been pregnant and/or is still lactating, but which may not be the actual mother of the recipient. The term “a mother” does not infer that the mother is related in a parent/child manner to the recipient as mentioned herein unless specifically stated otherwise.

The terms “prevention” and “prevent” with respect to a disease as used herein means a reduction, alleviation and/or amelioration of the adverse effects of a disease or disorder, or the complete cessation of a disease or disorder from occurring. In some embodiments, the terms “prevention” and “prevent” as used herein may mean the cessation of a disease or disorder from occurring.

In some aspects, the compositions and methods of the present invention may lead to the prevention of specific diseases in the recipient beyond infancy and may even lead to the lifelong prevention of specific diseases in the recipient.

In one aspect of the present invention preferably the immune modulator composition and/or the pharmaceutical composition additionally comprises at least one antigen or antigenic determinant, wherein said antigen or antigenic determinant is a viral antigen of bovine papilloma viruses.

Typically, an immune modulator composition or a pharmaceutical composition invention may be an immune enhancer.

In the alternative or in addition to the diseases mentioned above, compositions of the present invention may be used in the manufacture of a medicament to prevent, alleviate and/or reduce in an infant the “deleterious response to self”, i.e. autoimmune response, associated with many diseases and to reduce susceptibility to specific autoimmune diseases such as primary diabetes mellitus, psoriasis, rheumatoid arthritis of childhood and psychiatric diseases of the schizophrenia-depressive psychosis axis.

### Immune Modulator

The term “immune modulator”, as used herein, means a substance which modulates a cellular immune system of a recipient.

Suitably the “immune modulator” may enhance the cellular immune system of a recipient.

Likewise the term “modulating the immune response” as used herein means an alteration, which may be an increase or a decrease, to the immune response elicited by the infant.

Preferably the immune response is increased (i.e. enhanced). The term “modulates the immune response” should be construed accordingly.

Suitably, such modulation may correct potential dysregulation of the immune response.

The term “enhance the immune response” as used herein means the infants immune response is increased, for instance to effect a benefit to the health and/or growth of said infant. Preferably, the enhanced immune response is an enhanced cellular immune response.

In accordance with the present invention, immune enhancers may be used, for example, for the prevention of an immune system imbalance in an infant, or for enhancing the immune system of an infant for example of a mammal, particularly of livestock or of humans.

The subject may be treated with an immune enhancer (e.g. such as whole cells of the present invention) by consumption in specially designed food or in animal feeds, for example pig animal feeds supplemented with the bacteria of the present invention.

The immune enhancers may also be administered by other routes—such as direct injection.

In one embodiment of the present invention the subject is treated with whole cells of a bacterium of the present invention prior to and/or during pregnancy.

Suitably the subject may be treated in accordance with the present invention when it is young itself, for example when it is still in the neonatal period itself. Without wishing to be bound by theory, the administration of the composition in accordance with the present invention to a subject whilst the subject is still an infant (i.e. in its neonatal period, namely the period just after its birth), may enhance the subject's ability to transfer immunity to an infant of which the subject is a mother. In this regard, administering the composition according to the present invention to a first generation during the neonatal period may have an enhanced effect on the immunity of the second generation.

One embodiment of the present invention relates to a recipient being exposed to a bodily fluid of a subject treated with a composition of the present invention, wherein such exposure to the bodily fluid is whilst the subject is pregnant with the recipient. Without wishing to be bound by theory, it is thought that administration of the composition according to the present invention to a subject prior to and/or during pregnancy will result in a component(s) in the blood (for example) of the subject that is/are capable of modulating the immune response of a recipient. During pregnancy such component(s) may cross the placenta and the exposure of the recipient to such component(s) results in the modulation of the immune system of the recipient. Suitably the exposure may occur whilst the recipient's immune system is developing.

Another embodiment of the present invention relates to a recipient being exposed to a bodily fluid of a subject administered with a composition according to the present invention whilst the recipient is an infant. In this embodiment the “bodily fluid” may be milk. Suitably the milk or one or more components of the milk is capable of modulating an immune response in the infant fed on the milk. Without wishing to be bound by theory, administration of the composition according to the present invention to a subject prior to lactation results in a component(s) in the milk of the subject. A recipient may be exposed to such a component(s) as an infant by ingestion of the milk or part thereof, resulting in the immune system of the recipient being modulated. For the avoidance of doubt, in this embodiment the subject need not be the actual mother of the recipient, i.e. the subject may be a wet-nurse.

In yet another embodiment of the present invention the immune system of a recipient may be modulated by exposure to bodily fluid of the subject both during pregnancy and through feeding in the neonatal period.

Suitably, the recipient being exposed to such bodily fluid or bodily fluid component(s) during infancy may cause an immune modulating effect on the recipient that extends beyond infancy and throughout the life of the recipient.

For example, it has been shown that vaccination with vaccinia early on in life can significantly prolong the survival of patients with malignant melanoma (Kölmel et al, Prior immunisation of patients with malignant melanoma with vaccinia or BCG is associated with better survival. An European Organization for Research and Treatment of Cancer cohort study on 542 patients. European Journal of Cancer, 2005, 41: 118-125)

Preferably, the bacteria are killed so as to avoid the difficulties of maintaining live products. Preferably, the bacterium according to the present invention is killed by heat-treatment thereof, for example, heat-treatment in an autoclave at 121° C. for 15 minutes.

Other suitable treatments for killing the bacterium may include ultraviolet or ionising radiation or treatment with chemicals such as phenol, alcohol or formalin. Suitably the ionising radiation may be carried out by exposure to 2.5 Mrads from a Co₆₀ source

Preferably, a recipient as an infant is immunised though a subject against one or more of malaria, trypanosomiasis, leishmaniasis and toxoplasmosis.

In one aspect, the composition of the present invention may result in the down regulation of a Th2 response in a recipient.

In another aspect, the composition of the present invention may result in the up regulation of a Th1 response in a recipient.

Suitably, the composition of the present invention may result in the downregulation a Th2 response and up regulate a Th1 response in a recipient.

Alternatively, the composition of the present invention may result in the up regulation of a Th1 response whilst not affecting a Th2 response in a recipient.

Alternatively, the composition of the present invention may result in the downregulation a Th2 response, whilst also down regulating a Th1 response in a recipient.

Alternatively, the composition of the present invention may result in the upregulation of a Th2 response, whilst also upregulating a Th1 response in a recipient.

By way of example only, administering the subject with any one or more of the following organisms: Rhodococcus ruber, Rhodococcus rhodocrous, Dietzia maris and Gordona terrae may result in an enhanced Th1 response, without changing the Th2 response a recipient.

By way of example only, administering the subject with any one or more of the following organisms: Gordona bronchialis, Tsukamurella inchonensis, Gordonia amarae and Nocardia asteroides may result in an enhanced the Th1 response, or leave the Th1 response unchanged and down-regulated the Th2 response in a recipient.

By way of example only, administering the subject with Rhodococcus coprophilus may result in down-regulation of both Th1 and Th2 responses in a recipient, suitably administering the subject with Rhodococcus coprophilus may result in a strong down-regulation of both Th1 and Th2 responses in a recipient.

In one embodiment of the present invention a recipient may be a pig. Suitably, administering the subject (e.g. a donor pig, such as a mother pig) with Rhodococcus coprophilus may result in bodily fluid and/or bodily fluid components that prevent or protect the recipient (e.g. another pig) from post-weaning multisystemic syndrome (PMWS) and/or porcine dermatitis and nephropathy syndrome (PDNS). Suitably, the recipient (e.g. the other pig) may be protected through infancy (e.g. as a piglet) and beyond.

The term “protect” as used herein means that the recipient is less susceptible to the disease/disorder as compared with an animal not exposed to a bodily fluid or bodily fluid component of a subject that has been administered with a bacterium of the present invention and/or that the recipient is more able to counter or overcome the disease as compared with an animal not exposed to a bodily fluid or bodily fluid component of a subject that has been administered with a bacterium of the present invention.

Preferably, the bacterium of the present invention is used to modulate (preferably enhance) the protected immune response in a recipient (preferably an infant). By this we mean that the recipient may be protected against a disease and/or a disorder, i.e. may be less susceptible to said disease and/or disorder. Alternatively or in addition, the disease and/or disorder should it occur in the recipient may be ameliorated compared with the disease and/or disorder in an untreated animal. The term ameliorated for instance, as used herein may mean that the disease and/or disorder causes less damage (such as less tissue injury) and/or there may be less parasitemias and/or bacteria and/or viruses for instance in the blood of the recipient compared with an untreated animal.

### Bacteria of the Present Invention

For ease of reference the terms “whole cells of bacteria of the present invention” and “bacteria of the present invention” as used herein are shorthand for whole cells of a bacterium from a genus of aerobic organisms in the order of Actinomycetales.

The term “whole cells from a bacterium of a genus of aerobic organisms in the order of Actinomycetales” as used herein encompasses whole cells of one or more strains of bacteria Suitably, where the whole cells are from more then one stain of bacteria, said strains may be from one or more genera. And suitably, where said whole cells are from more than one genus, said genera may be from one or more families. Thus, the term encompasses whole cells from a particular strain (for example, whole cells of a particular strain of Tsukamurellla inchonensis) and whole cells from bacteria, of a genus of aerobic organisms, from different families (for example, whole cells of strains from Tsukamurella inchonensis or M. obuense, which are from the families of Tsukamurellaceae and Mycobacteriaceae).

In one embodiment, suitably the aerobic organism in the order Actinomycetales for use in accordance with the present invention may be from the genus Mycobacterium (such as M. vaccae or M. obuense).

In another embodiment, suitably the aerobic organism(s) in the order Actinomycetales for use in accordance with the present invention may be Nocardioform actinomycetes (such as bacteria mentioned in Group 22 of Bergy's Manual of Determinative Bacteriology, Ninth Edition. Such as, for example, mycolic-acid containing bacteria).

Preferably, the aerobic organism(s) are mycolic acid-containing bacteria (such as bacteria in Group 22 subgroup 1 of Bergy's Manual of Determinative Bacteriology, Ninth Edition. Such as, for example, Gordonia, Rhodococcus, Norcardia and Tsukamurella).

Preferably, the aerobic organism(s) may be from one or more of the following genera: Gordonia (such as G. bronchialis, G. amarae, G. sputi and G. terrae, preferably G. bronchialis); Rhodococcus (such as Rhodococcus ruber (previously known as Nocardia rubra), R. rhodnii, coprophilus, R. opacus and R. erythopolis, preferably from R. coprophilus); Norcardia (such as Norcardia asteroides and N. brasiliensis) and Tsukamurella (such as T. inchonensis and T. paurometabola, preferably from T. inchonensis).

In yet another embodiment, suitably the aerobic organism(s) in the order Actinomycetales for use in accordance with the present invention may be from a genus or genera that contain mycolic acid as a component of the cell wall. Examples of such genera include: Gordonia, Mycobacterium, Dietzia, Rhodococcus, Norcardia and Tsukamurella.

Preferably, the aerobic organism(s) in the order Actinomycetales for use in accordance with the present invention is/are from one or more of the following genera: Gordonia (such as G. bronchialis, G. amarae, G. sputi and G. terrae, preferably G. bronchialis); Myobacterium (such as from M. vaccae and M. obuense, preferably from M. obuense); Dietzia (such as D. maris); Rhodococcus (such as from Rhodococcus ruber (previously known as Nocardia rubra), R. rhodnii, R. coprophilus, R. opacus and R. erythopolis, preferably from R. coprophilus); Norcardia (such as from Norcardia asteroides and N. brasiliensis) and Tsukamurella (such as T. inchonensis and T. paurometabola, preferably from T. inchonensis).

Suitably the aerobic organism(s) may be from the genus Gordonia. Preferably, the aerobic organism(s) is/are from one or more of the following: G. bronchialis, G. amarae, G. sputi and G. terrae, preferably from G. bronchialis.

Suitably, the genus “Gordonia” as used herein may be referred to as “Gordona”. These genus names are used interchangeably herein.

Suitably the aerobic organism(s) may be from the genus Tsukamurella. Preferably, the aerobic organism(s) is/are from T. inchonensis and/or T. paurometabola, preferably from T. inchonensis.

Suitably the aerobic organism(s) may be from the genus Mycobacterium. Preferably, the aerobic organism(s) is/are from M. vaccae and/or M. obuense, preferably from M. obuense.

A M. obuense strain for use in accordance with the present invention has been deposited by BioEos Limited of 67 Lakers Rise, Woodmansteme, Surrey, SM7 3LA under the Budapest Treaty on the International Recognition of the Deposit of Microorganisms for the purposes of Patent Procedure at the National Collection of Type Cultures (NCTC), Central Public Health Laboratory, 61 Colindale Avenue, London, NW9 5HT) on the 14 Jul. 2005, under Accession Number NCTC 13365.

Suitably the aerobic organism(s) may be selected from the genus Rhodococcus. Suitably, the aerobic organism(s) may be selected from any one or more of the following species: Rhodococcus Tuber (previously known as Nocardia rubra), R. rhodnii, R. coprophilus, R. opacus and R. erythopolis, preferably from R. coprophilus.

Suitably the aerobic organism(s) may be selected from the genus Dietzia. Suitably, the aerobic organism(s) may be selected from the species Dietzia maris.

Suitably the aerobic organism(s) may be selected from the genus Norcardia. Suitably, the aerobic organism(s) may be selected from any one or more of the following species: Norcardia asteroides and/or N. brasiliensis.

The term “whole cell”, as used herein, means a bacterium which is intact, or substantially intact. In particular, the term “intact” as used herein means a bacterium which is comprised of all of the components present in a whole cell, particularly a whole, viable cell, and/or a bacterium which has not been specifically treated to remove one or more components from it. By the term “substantially intact” as used herein it is meant that although the isolation and/or purification process used in obtaining the bacterium may result in, for example, a slight modification to the cell and/or in the removal of one or more of the components of the cell, the degree to which such a modification and/or removal occurs is insignificant. In particular, a substantially intact cell according to the present invention has not been specifically treated to remove one or more components from it.

For the avoidance of doubt, when it is the case that the bacterium is killed prior to use, for example by heat-treatment, such heat treatment may inactivate or destroy constituents of the bacterium. Such a killed, for example heat treated, bacterium may still be considered as a substantially intact whole cell in accordance with the present invention.

The modulation of a cellular immune response in a recipient as an infant (caused by administration of said whole cell of said bacterium to a subject) may be advantageously long lasting as compared with the response in an animal as an infant elicited by administration of an individual component of the bacterium to a subject.

Preferably, the composition a whole cell of a bacterium according to the present invention comprises more than one whole cell, and more preferably comprises a plurality of whole cells.

Suitably the bacteria for use in the present invention may be killed prior to use.

### Subject

The term “subject”, as used herein, means an animal that is to be or has been administered with a composition according to the present invention. Preferably, the subject is a mammal, including for example livestock and humans. In some aspects of the present invention, the subject may suitably be a human.

The term “livestock”, as used herein refers to any farmed animal. Preferably, livestock is one or more of horses (including race horses), poultry, pigs (including piglets), sheep (including lambs), cows or bulls (including calves). More preferably, livestock means pigs—including piglets.

Suitably, the subject of the present invention may be the parent of the recipient.

In another embodiment, preferably the subject is administered with a whole cell of a bacterium of the present invention prior to and/or during pregnancy, preferably during pregnancy.

### Recipient

The term “recipient” as used herein relates to an animal (throughout its life from conception to death unless stated otherwise) that is exposed to a bodily fluid of a subject during infancy. Preferably, the recipient is a mammal, including for example livestock and humans. In some aspects of the present invention, the recipient may suitably be a human.

The present invention is predicated on the surprising finding that exposure of a recipient to a bodily fluid or bodily fluid component of a subject that has been administered with a composition of the present invention leads to a modulation in the immune response of the recipient that may have life long effects.

### Infant

The term “infant” as used herein refers to the recipient during pregnancy and before birth (e.g. as a foetus), as well as the recipient during and/or after the neonatal period, i.e. the period immediately after birth.

In accordance with the present invention, preferably it is as an infant that the recipient is exposed to a bodily fluid of a subject.

Preferably, the infant is the young of an animal, preferably a mammal—including, for example, livestock or humans.

In some aspects of the present invention an infant may suitably be a human infant.

### Bodily Fluid

The term “bodily fluid” as used herein includes for example milk and/or blood and/or a component thereof. The use of the term “bodily fluid” as used herein encompasses “bodily fluid component(s)” and reference to “bodily fluid” herein may refer to a bodily fluid component rather than the whole bodily fluid. The bodily fluid component(s) is/are capable of eliciting a modulation in the immune response of an infant. Preferably such components are capable of enhancing the immune response of an infant.

By way of example only and without wishing to be bound by theory, the bodily fluid component(s) may be:
1. the whole cell of a bacterium of the present invention;
2. antibodies raised against said whole cell of a bacterium of the present invention;
3. one or more further constituents capable of eliciting a modulation in the immune of an infant;
4. plasma; and/or
5. serum.

Suitably in one embodiment of the present invention where the subject is pregnant with the infant such “bodily fluid components” may be able to cross the placenta to elicit an immune response in the infant.

### Adjuvants

The term ‘adjuvant’ as used herein means an entity capable of augmenting or participating in the influencing of an immune response. An adjuvant is any substance or mixture of substances that assists, increases, downregulates, modifies or diversifies the immune response to an antigen.

Hence, the composition of a bacterium of the present invention for use in the methods and uses of the present invention may be described as an adjuvant.

Suitably the composition comprising a whole cell of a bacterium of the present invention may be administered to the subject in combination with one or more adjuvants, which enhance the effectiveness of the immune modulator composition. Examples of additional adjuvants which, may be effective include but are not limited to: aluminium hydroxide, aluminium phosphate, aluminium potassium sulphate (alum), beryllium sulphate, silica, kaolin, carbon, water-in-oil emulsions, oil-in-water emulsions, muramyl dipeptide, bacterial endotoxin, lipid X, Corynebacterium parvum (Propionobacterium acnes), Bordetella pertussis, polyrbonucleotides, sodium alginate, lanolin, lysolecitin, vitamin A, interleukins such as interleukin 2 and interleukin-12, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.). Only aluminium hydroxide is approved for human use. Some of the other adjuvants have been approved for clinical trials.

Suitably, the compositions of the present invention may also prevent the disadvantageous effects of adjuvants in multiple vaccination programmes.

### Antigen

As used herein, an “antigen” means an entity which, when introduced into an immunocompetent host, modifies the production of a specific antibody or antibodies that can combine with the entity, and/or modifies the relevant Th response, such as Th2 and/or Th1. The antigen may be a pure substance, a mixture of substances or soluble or particulate material (including cells or cell fragments or cell sonicate). In this sense, the term includes any suitable antigenic determinant, cross reacting antigen, alloantigen, xenoantigen, tolerogen, allergen, hapten, and immunogen, or parts thereof, as well as any combination thereof, and these terms are used interchangeably throughout the text.

The term “antigenic determinant or epitope” as used herein refers to a site on an antigen which is recognised by an antibody or T-cell receptor, or is responsible for evoking the T-helper cell response. Preferably it is a short peptide derived from or as part of a protein antigen. However the term is also intended to include glycopeptides and carbohydrate epitopes. The term also includes modified sequences of amino acids or carbohydrates which stimulate responses which recognise the whole organism.

Suitably the composition comprising a whole cell of a bacterium of the present invention may be administered to the subject in combination with one or more antigens and/or antigenic determinant.

It is advantageous if the antigenic determinant is an antigenic determinant of the infectious agent which causes the infectious disease. For example, in the prevention and treatment of Chagas' disease in a recipient an antigen or antigenic determinant that may be used in combination with whole cells of a bacterium of the present invention may suitably by Trypanosoma cruzi.

A “preventative” or “prophylactic” vaccine is a vaccine which is administered to naive individuals to prevent development of a condition, such as by stimulating protective immunity.

### Administration

Typically, a physician will determine the actual dosage of a vaccine, immune modulator composition and pharmaceutical composition to be administered to a subject which will be most suitable to result in an immune modulation in an infant and it will vary with the age, weight and response of the particular subject. The dosages below are exemplary of the average case. There can, of course, be individual instances where higher or lower dosage ranges are merited.

Preferably, the actual dosage that is used results in minimal toxicity to the subject.

The compositions to be used in the methods and uses of the present invention may be administered to the subject by direct injection.

The composition may be formulated for parenteral, mucosal, intramuscular, intravenous, subcutaneous, intraocular, intradermal or transdermal administration.

Suitably, the composition according to the present invention may be administered at a dose of 10³-10¹¹ organisms, preferably 10⁴-10¹⁰ organisms, more preferably 10⁶-10−5×10⁹ organisms, and even more preferably 10⁶-10⁹ organisms. Typically, the composition according to the present invention may be administered at a dose of 10⁸-10⁹ bacteria for human and animal use.

If the compositions of the present invention are to be administrated as immune enhancers, then 10³-10¹¹ organisms per dose, preferably 10⁴-10¹⁰ organisms per dose, more preferably 10⁶-10−5×10⁹ organisms per dose, and even more preferably 10⁶-10⁹ organisms per dose, and even more preferably, 10⁸-10⁹ bacteria per dose for human and animal use may be administered at regular intervals.

As will be readily appreciated by a skilled person the dosage administered will be dependent upon the subject to which the dose is being administered.

The term “administering” as used herein refers to administration of bacteria of the present invention for the purposes of providing a medicament. Preferably, “administering” relates to administration to a subject for the purpose of modulating the immune system of a recipient. In other words, in one embodiment the term “administering” means that the bacteria is given (preferably as a medicament) to the subject, i.e. “administering” does not encompass the situation where the subject per se may comprise or acquire the bacteria naturally.

The term “administered” includes delivery by delivery mechanisms including injection, lipid mediated transfection, liposomes, immunoliposomes, lipofectin, cationic facial amphiphiles (CFAs) and combinations thereof, or even viral delivery. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestible solution; a parenteral route where delivery is by an injectable form, such as, for example, an intravenous, intramuscular, intradermal or subcutaneous route.

Preferably, in the present invention, administration is by injection. More preferably the injection is intradermal.

Preferably, in the present invention, administration is by an orally acceptable composition.

For vaccination the composition can be provided in 0.1 to 0.2 ml of aqueous solution, preferably buffered physiological saline, and administered parenterally, for example by intradermal inoculation. The vaccine according to the invention is preferably injected intradermally. Slight swelling and redness, sometimes also itching may be found at the injection site. The mode of administration, the dose and the number of administrations can be optimised by those skilled in the art in a known manner.

### Infectious Diseases

Compositions capable of modulating, in particular stimulating (i.e. inducing or enhancing) T cell proliferation and/or differentiation or of preventing the induction of or reversing T cell energy in a recipient may be used generally to boost or induce T cell immune responses in a recipient.

The bodily fluid compositions of the present invention may be used generally to prevent infectious diseases—such as viral or bacterial—in a recipient. Suitably, the bodily fluid compositions of the present invention may protect the recipient from an infection throughout the recipient's life. Preferably the bodily fluid compositions of the present invention may protect the recipient from an infection throughout infancy.

Suitably, the compositions of the present invention may be used to prevent parasitic infections, e.g. malaria, leishmaniasis, toxoplasmosis and trypanosomiasis). Suitably, the compositions of the present invention may be used to prevent viral infections, for example infections caused by papilloma viruses, including equine sarcoid, genital warts and dysplasia of the uterine cervix that precedes carcinoma of the cervix, HIV, mycoses, chlamydia and herpes.

In one aspect of the present invention the infection is preferably HIV.

In Yet Another Aspect of the Present Invention the Infection is Caused by T. Cruzi. Equine Sarcoid

The compositions of the present invention may also be used to prevent equine sarcoid in a recipient (e.g. a horse). Preferably, the compositions of the present invention may also be used to prevent equine sarcoid in a recipient during infancy (e.g. in a foal).

Equine sarcoid is the commonest skin neoplasm of horses and is associated with infection with bovine papilloma viruses 1 and 2 (Chambers et al J. Gen. Virol. 2003: 84: 1055-1062). This condition is currently without an effective treatment, although surgery, non-specific immune treatment and cytotoxic drugs may all have some effect.

The compositions of the present invention are preferably administered in the same lymph node drainage area as a lesion or neoplasm.

### PMWS and PDNS

Post-weaning multisystemic wasting syndrome (PMWS) affects piglets post-weaning from 4 to 16 weeks of age (15-50 kg). Typically PMWS affects piglets one to two weeks after weaning and is very different from the wasting/poor weaner who fails to eat or drink adequately after weaning. PMWS piglets are weaners which have started to grow and then collapse quickly and often have an extremely poor response to antibiotics.

Porcine dermatitis and nephropathy syndrome (PDNS) affects pigs from 8 to 18 weeks of age and the most obvious signs are red-purple blotches on the skin, which become brown and crusted after a few days. Pigs are lethargic and may have swollen legs resulting from their nephropathy. This syndrome, also, responds poorly to antibiotics.

The causal agents of both PMWS and PDNS are at present unknown. The most likely suspect in both syndromes is a pig circovirus “type II”, which is antigenically distinct from widely distributed normal non-pathogenic pig circovirus “type I”. Circovirus II (PCV II) has been identified on UK farms serologically. PDNS, which is thought to be an immune complex mediated disease, may also involve bacteria in its aetiology, though the part that they play is not clear

Suitably the compositions of the present invention may be used in the manufacture of a medicament for the prevention of PMWS and/or PDNS in a recipient, preferably a piglet. Thus, preferably the composition is administered to a sow.

### Allergy

The compositions of the present invention may also be used to prevent allergies (e.g. a food allergy including a nut allergy, asthma including allergic asthma, hayfever, allergic dermatitis (eczema), allergies to plant contact or ingestion, stings—such as nettle and insect stings, and allergies to insect bites—such as midges for instance Culicoides (which causes Sweet Itch in horses and fleas) in a recipient.

Thus, the allergy may involve skin (as in allergic dermatitis), the respiratory tract (as in asthma and COPD), the gut (as in food intolerance) or the conjunctival sac (as in allergic conjunctivitis).

Preferably the compositions of the present invention may be used to treat food allergies and/or asthma.

Recent studies have highlighted the influence of fetal/maternal interactions on the development of asthma. In this regard, Lima et al, (Journal of Immunology, 2005, 175:3554-3559) discloses modulation of the induction of lung and airway allergy in the offspring of IFN-γ-treated mother mice.

Sweet Itch is one of the commonest skin diseases seen in horses, particularly in wild horses and/or ponies. About 3% of horses in the U.K. are affected to some degree. Most horses show signs between 1 and 4 years of age and the condition generally worsens during summer. Certain breeds are particularly prone to the disease. Shires, Hackneys and Welsh and Icelandic ponies have all been suggested as susceptible breeds. Sweet Itch is caused by hypersensitivity to the bites of the tiny fly Culicoides. In the UK, the fly is present from April to October but peaks in numbers in May to September. The flies feed on the horse at specific sites usually around the tail head and under the mane. There are 20 species of Culicoides present in the UK and some feed underneath the horses' abdomen.

### Heaves/COPD

The compositions of the present invention may also be used to prevent heaves and/or COPD (Chronic Obstructive Pulmonary Disease) in an infant.

Suitably, the compositions of the present invention may be used in the manufacture of a veterinary medicament for the treatment or prevention (preferably prevention) of heaves and/or COPD.

Heaves is an equine lung disease with similarities to human asthma and COPD. The clinical signs in the horse are initiated by an allergic response to the particles in hay dust in lungs already damaged with a degree of fibrosis. It is most often seen in older horses (greater than six years old) that are stabled during the winter months. Hay contains microorganisms—such as bacteria and fungi as well as tiny particles of feed grains, plants, faeces, dander, and pollen. These tiny particles become aerosolised in hay dust and elicit an allergic response and fibrosis when they are inhaled by horses with heaves. The primary microorganisms believed to be involved in the etiology of heaves are Aspergillus fumigatus, Thermoactinomyces vulgaris and Faenia rectivirgula. Both reduction of the bronchospasm of asthma and the fibrosis of COPD are within the scope of the patent.

### Autoimmune Diseases

The compositions of the present invention may be used to treat and/or prevent an autoimmune disease mechanistically related to poor T cell regulation and/or T cell dysregulation in a recipient. Examples of autoimmune diseases include one or more of the following: unwanted immune reactions and inflammation including arthritis, including rheumatoid arthritis, psoriasis, psoriatic arthropathy, vascular disorders, in particular a vascular disorder in which there is inflammation of the intima of the blood vessel, examples of vascular disorders are atheroma formation (otherwise known as arteriosclerosis), anterior uveitis and myointimal hyperplasia following angioplasty; thyroiditis, atherosclerotic heart disease, reperfusion injury, cardiac conduction disturbances, myocardial infarction, habitual abortion, retinitis pigmentosa, immune and inflammatory components of degenerative fundus disease, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, Guillaim-Barre syndrome, myasthenia gravis, graft rejection in cases of transplantation of natural or artificial cells, tissue and organs such as cornea, bone marrow, organs, lenses, pacemakers, natural or artificial skin tissue.

In more detail: Organ-specific autoimmune diseases include multiple sclerosis and inflammatory bowel diseases (Crohn's disease, ulcerative) for example.

Systemic autoimmune diseases include rheumatoid arthritis.

Vascular disorders include vascular disorders in which there is inflammation of the intima of the blood vessels.

Suitably, the vascular disorders according to the present invention may include any vascular disease or disorder which comprises an autoimmune element, for example one which is caused by an autoimmune response.

Suitably, vascular disorders according to the present invention may include one or more of Raynaud's disease and phenomenon, anterior uveitis, obliterative vascular disorder, atheroma formation (otherwise known as arteriosclerosis), arteritis, myointimal hyperplasia (natural or following angioplasty), inflammatory and autoimmune thickening of the intima and/or muscular layer of blood vessels, inflammatory blood vessel lesions, atherosclerotic heart disease, reperfusion injury, cardiac conduction disturbances, myocardial infarction.

Suitably, the graft rejection according to the present invention may be chronic graft rejection, particularly in the absence of an immunosuppressant. Thus, the composition according to the present invention may be used as a replacement for the conventional immunosuppressant administered prior to, during and/or after transplantation. The compositions according to the present invention may be used when transplanting natural or artificial cells, tissues and organs, such as one or more of the following: corneas, bone marrow, organs (e.g. kidney, liver), lenses, pacemakers, natural or artificial skin tissue, islet cells.

Preferably, the compositions of the present invention may be used to treat the following autoimmune diseases: a vascular disorder, arthritis, graft rejection and the immunological aspects underlying myointimal hyperplasia and atheroma formation.

### Stress

Stress is often presented as a symptom of modern living, the high pressure executive lifestyle, the consequences of which are widely perceived as leading to major pathological conditions such as gastric ulcers, hypertension, heart disease and strokes. Other major stressful events in life such as divorce, bereavement and moving house are seen as high risk factors for heart disease.

These are not misconceptions, the farming industry is well aware of the economic losses resulting from subjecting livestock to major stresses such as overcrowding, confinement and transportation leading to an increased susceptibility to infection and the precipitation of underlying pathology. Research by doctors and scientists is producing an increasing volume of published work showing definable stresses such as confinement can result in significant changes in endocrine (hormone) activity which subsequently can affect the body's immune functions. This can be noticeably demonstrated in major trauma stress (including surgical stress) in which the cell mediated immune response is dramatically paralysed Faist (1996).

Elenkov I J (1999) report recent evidence indicating that glucocorticoids and catecholamines, the end products of the stress system, and histamine, a product of activated mast cells, might selectively suppress cellular immunity, and favour humoral immune responses. This is mediated by a differential effect of stress hormones and histamine, on Th1/Th2 patterns and type 1/type 2-cytokine production. Thus, systemically, stress might induce a Th2 shift, while, locally, under certain conditions, it might induce pro-inflammatory activities through neural activation of the peripheral corticotrophin-releasing factor-mast cell-histamine axis.

Paik (2000) and Kay (2001) in independent studies of academic stress, examined the immunological profiles of students during non exam and exam periods. They report a significant reduction in IL-2 and interferon gamma production and an increase in IL-6. This indicates that the body's immune system responds to stressful episodes by a down regulation of Th-1 cytokines and a selective up-regulation of the Th2 cytokines.

Iwakabe (1998), using a mouse model of restraint stress, reports the skewing of the immune response towards Th2 dominant immunity.

This stress hormone induced switch towards Th2 immune imbalance is also reported in non major, chronic stress situations such as psychosocial stress amongst workers overwintering at the Australian National Antarctic Research Expedition stations, (Mehta (2000)). They also report an associated increase in latent virus reactivations.

Similar stress hormone and immunological changes are reported from chronic stress in care givers of dementia patients (Bauer (2000)) and in astronauts during the Euromir 95 mission (Norbiato (1998)). Of particular concern was the astronauts increased susceptibility to infection.

The body is designed to recover from stress and in acute stress clearly does as the risk of infection recedes with the patient's recovery from the major trauma.

Chronic stress however appears to maintain the Th2 dominated immune imbalance. This is a very serious consequence as all of the quoted authors allude to stress through the above mechanisms, possibly influencing the onset and/or course of infectious, inflammatory, allergic and neoplastic-diseases.

This consequence is further supported by Lawrence (2000).

A bodily fluid composition according to the present invention, which stimulates the Th1 response and down regulates Th2 may restore the healthy balance of the immune system and thus reduce the increased risk of serious illness associated with chronic stress.

Preferably, the compositions according to the present invention is used to treat and/or prevent (preferably prevent) stress, in particular major trauma stress, psychosocial stress and chronic stress in an infant.

Preferably, the compositions according to the present invention is used to treat and/or prevent (preferably prevent) stress in recipients, suitably such recipients may be humans and/or livestock.

### Immune System Imbalance

An immune system imbalance—such as an upregulation, downregulation or inappropriately regulated cellular immune response—may occur at any time in the life of a recipient. Suitably, compositions of the present invention may be used to modulate an immune system imbalance in a recipient. That is to say, the compositions according to the present invention may be used to treat and/or prevent (preferably prevent) an immune system imbalance in a recipient

### (a) In Children

Suitably, the composition may be used to modulate an immune system imbalance, in children, including babies, infants and juveniles. An immune system imbalance—such as an upregulation, downregulation or inappropriately regulated cellular immune response—may occur in children following vaccination, for example following childhood vaccinations. Such an immune system imbalance may result in conditions such as the onset of allergies, i.e. allergic dermatitis and allergic asthma.

With the aim of protecting children from infections, repeated injections against Diphtheria, Tetanus, Pertussis, Polio, Measles, Mumps and the Rubella are given. All of these are judged necessary and pressure is exerted by Health authorities to ensure that children are presented for vaccination at the appropriate time. However, most vaccinations given in early life contain an alum adjuvant, which has important immunological consequences. Alum is a potent stimulus to the Th2 pattern of response and the consequential immune dysregulation causes the child to become vulnerable to the development of allergies and possibly cancer for example.

It is possible to re-educate the immune system to a proper recognition, regulation and response both to self and to the rest of the world.

Suitably, the composition may also be used for the treatment or prevention (preferably prevention) of an adverse reaction to childhood vaccines—such as whooping cough vaccinations and the current MMR vaccinations—and/or consequences thereof.

Suitably, in one aspect of the present invention it is envisaged that a whole cell of the bacterium according to the present invention may be included in food preparations and/or may be supplied as a type of “remedy”, preferably orally.

### (b) Immune System Imbalance in the Elderly

An immune system imbalance—such as an upregulation, downregulation or inappropriately regulated cellular immune response, in particular downregulation, for example a deterioration of immune function—may occur in older people, generally in excess of 60 years. In elderly people, a downregulation in the cellular immune response is generally referred to as immunosenescence. Typically, the deterioration of immune function may lead to increased susceptibility to infectious diseases and neoplasia for example. The number of old people as a proportion of the population is dramatically increasing and geriatric medicine is becoming an important aspect of clinical practice. It is not surprising therefore that research has focused on the mechanisms of immunosenescence and the links between the health of the immune system and longevity. Goronzy (2001), examined the varying efficacy of influenza vaccination in the elderly. In this study, only 17% of subjects showed a rise in titre to all 3 haemagglutinins (successful vaccination) 1 month following vaccination and that 46% showed no demonstrable response at all. It was proposed that responsiveness to influenza vaccination is a useful biological marker of immunosenescence. A number of researchers have studied various aspects of the immune function in the elderly. For example, Lio (2000) studied cytokine responses, Solana (2000) studied NK and NKt cells, and Ginaldi (1999) suggested that a Th1 to Th2 cytokine production shift and an increased production of proinflammatory cytokines could explain many aspects of age-associated pathological events, such as atherosclerosis and osteoporosis. Accordingly, a non-pathological stimulation of the immune system which drives the cytokine response away from the proinflammatory Th2 towards Th1 is required. Preferably, such an immune modulator reduces the mortality from acute infection, counters the onset and reduces the morbidity of age related autoimmune disease and possibly reduces the rate of neoplastic disease, all of which are associated with immunosenescence.

Therefore the present invention may encompass the administration of a composition of the present invention to a subject in order to reduce and/or prevent immune system imbalance in an elderly recipient. Without wishing to be bound by theory, the exposure of a recipient to a bodily fluid component of a subject administered with a composition according to the present invention whilst the recipient is an infant, may reduce and/or prevent immune system imbalance in the recipient's later life, e.g. once the recipient has become elderly.

### Enhancing the Immune System

The compositions of the present invention may be for enhancing the immune system in a recipient, preferably a mammal recipient, more preferably a livestock recipient, which may result in for example, enhancement (e.g. promotion) of growth and/or an increase in the efficiency of feed utilisation and/or a generally increased well-being (i.e. the overall health of the infant is improved) in the recipient. The overall health of a recipient can be determined by one or more of the following parameters for example: weight data (with weight gain being a positive determinant), alertness (with full alert being a positive determinant), movement (with energetic movement as opposed to lethargic movement being a positive determinant) and sickness (with reduced amount of sickness being a positive determinant).

The present invention also contemplates the bacteria of the present invention being administered to a subject in combination with known probiotic bacteria, for modification of the cellular immune response in the recipient.

Commercially at present antibiotics are commonly used as dietary enhancing feed additives (or growth promoters) and are incorporated into animal feed. However, the EU, is expected to introduce a complete ban on the non-clinical use of antibiotics in animal husbandry. Therefore, the market requires effective alternatives.

An advantage of the present invention is that the uses and method described herein may be used (optionally together with good animal husbandry practices) as a replacement to dietary enhancing feed additive (or growth promoters).

### Cancer

Suitably, the compositions according to the present invention are used to modulate a cellular immune response to treat and/or prevent (preferably prevent) cancer in an infant. In particular it is envisaged that the compositions according to the present invention may be used to protect an infant against developing and/or the progression of a cancer. In particular, the infant with a modulated cellular immune response may be less susceptible to the development of cancer.

In particular, during cancer growth an unregulated increase in Th2 is observed.

Cancer is a disease that affects many people, with 65 percent of cases occurring in those over 65. As the average life expectancy in the UK has almost doubled since the mid-nineteenth century, the population at risk of cancer has grown Death rates from other causes of death, such as heart disease, have fallen in recent years while deaths from cancer have remained relatively stable. The result is that 1 in 3 people will be diagnosed with cancer during their lifetime and 1 in 4 people will die from cancer.

Examples of cancer include bladder, brain tumour, breast cancer, cervical cancer, colon and rectal cancer, adenocarcinoma, endometrial cancer, oesophageal cancer, kidney cancer, leukaemia, liver cancer, lung cancer, melanoma, myeloma, non-Hodgkin's lymphoma, ovarian cancer, pancreatic cancer, prostate cancer, sarcoma, soft tissue and stomach cancer.

In addition, persistent smoking of tobacco, and to a lesser, extent passive smoking, has been associated with carcinomas of the parts directly in contact with smoke, oropharynx, trachea, lungs, esophagus and stomach. As well as these, distant tumours such as those of the kidney, bladder, pancreas, liver and myeloid leukaemia may be increase by smoking of tobacco. In the present invention, it is envisaged that compositions according to the present invention could be administered to smokers of tobacco in an attempt to reduce the recipients risk of developing carcinomas associated with tobacco smoking.

Suitably, the cancer may be an adenocarcinoma, carcinoma, leukaemia or a melanoma

Preferably the cancer is an adenocarcinoma or a melanoma. Preferably, the cancer is an adenocarcinoma, Suitably, the cancer may be virally related cancers such as cervical cancer for example. Without wishing to be bound by theory, in some instances it has been found that an infection caused by papilloma viruses, such as dysplasia of the uterine cervix, precedes carcinoma of the cervix. Thus, cervical cancer is herein considered a “virally related cancer”. However, the term “virally related cancer” as used herein means any cancer which may be caused by or related with a viral infection.

### Post-Operative Recovery, Stress and Infection

The compositions of the present invention may be used to treat or prevent (preferably prevent) post-operative, stress and infection in a recipient.

For the avoidance of doubt, the term “post-operative stress” as used herein may include (and preferably includes) the stress associated with the administration of anaesthetics.

Following any major operation a number of situations potentially arise:—

Stresses associated with a surgical operation include one or more of the following: apprehension before the operation, stress to the tissues due to the operative procedures, the pain usually accompanying recovery, worry about the significance of operative findings.

These kinds of stress are associated with the deviation of T-cell function towards Th2.

Immunosuppressive effects of premedication and anaesthetics, which may persist for days or weeks after the operation itself may be reduced by compositions of the present invention.

In addition, exposure of cut flesh to direct infection at the time of operation and of the wound to infection in the recovery room and wards prior to leaving hospital is also a problem.

A combination of these factors exposes the patient to a series of potential bacterial infections, which:—

Since the patient is hospitalised, include such notorious hospital-associated infections as those with methicillin-resistant-Staphylococcus aureus (MRSA). Operations on the bowel expose the patient to gram-negative infections due to exposure of cut tissues to bowel contents. Operations on the lower limbs are also subject to infections with normal members of the gut flora.

Minor infections of the wound delay healing and increase the chances of contracting more serious infections.

To counteract these influences, immune regulation towards Th1 and a down-regulation of Th2 a result of the application of the invention, should do one or more of the following: increase non-specific resistance to post-operative bacterial infections;

aid in wound healing and/or reduce stress.

### T Helper Cells

The term ‘Th1’ as used herein refers to a type 1 T-helper cell (Th1). The term may also be used herein to refer to the response mediated by or through such a cell type. Such a response may include one or more of the secretion of Interleukin-2 (IL-2), the secretion of Interferon-gamma (IFN-γ), activation of macrophages, activation of cytotoxic T-cells, or any other Th1-associated event. Thus, the term ‘Th1’ may include Th1 cell(s) as well as the immune response(s) which such cell(s) produce.

The term ‘Th2’ as used herein refers to a type 2 T-helper cell (Th2). The term may also be used herein to refer to the response mediated by or through such a cell type. Such a response may include one or more of the secretion of Interleukin-4 (IL-4), the secretion of the splice variant interleukin IL-462, the secretion of Interleukin-5 (IL-5), increase in levels of cell determinant 30 (CD30) on lymphocytes, increase in levels of Immunoglobulin-E (IgE) in the blood or eosinophils in the blood, or any other Th2-associated event. Thus, the term ‘Th2’ may include Th2 cell(s) as well as the immune response(s) which such cell(s) produce. Excessive antibody production with poor cellular responses are a characteristic of Th2 mediated responses.

It is known that various conditions may result in or from an unregulated or inappropriately regulated cellular immune response, in particular in the activation and/or proliferation of Th1 and/or Th2, which if left unregulated or inappropriately regulated has been found to result in one or more detrimental effects on the recipient.

In particular, such an unregulated or inappropriately regulated cellular immune response has been found to occur following vaccination, e.g. following childhood vaccinations, and is thought to result in conditions such as the onset of allergies, i.e. allergic dermatitis and allergic asthma. By way of example, Lewis D Curr Opin Immunol 2002; 14: 644 report that Th2 immune responses mediated by the secretion of IL-4, IL-5 and IL-13 are key in the pathogenesis of atopic disorders, including allergen-induced asthma, rhinoconjunctivitis and anaphylaxis. Although such responses are downregulated to some degree by conventional specific immunotherapy, this approach is only partially effective and has a substantial risk of adverse effects. Many strategies for immunotherapeutic prophylaxis and for treatment of atopic diseases have been devised on the basis of mouse allergy models, including the downregulation of Th2 responses by the induction of regulatory T cell activity, Th2 to Th1 immune deviation, Th1 crossregulation of Th2 immune responses, energy and immunosuppressive cytokines. Choi & Koh Ann Allergy Asthma Immunol 2002; 88: 584-91 examined whether BCG vaccination of adult patients with asthma, a Th2-associated allergic disease, is clinically effective. It was shown that BCG vaccination improved lung function and reduced medication use in adults with moderate-to-severe asthma. This amelioration was accompanied by a suppressed Th2-type immune response, suggesting that BCG vaccination might be an effective therapeutic modality against asthma von Hertzen J Allergy Clin Immunol 2002; 109: 923-8 outlined the possibility that prolonged maternal stress associated with sustained excessive cortisol secretion could affect the developing immune system—especially Th1/Th2 cell differentiation which may further increase the susceptibility to asthma and atopy in genetically predisposed individuals.

In addition, an unregulated or inappropriately regulated cellular immune response has been observed during disease progression. In particular during cancer growth an unregulated increase in Th2 is observed. By way of example, Maraveyas et al. Ann Oncol 1999; 10: 817-24 have studied the efficacy of SRL 172 vaccine in patients with cancer i.e. advanced stage IV (AJCC) malignant melanoma. Induction of intracellular cytokines (IL-2 and INF-gamma) in peripheral blood lymphocytes (PBLCs) from these patients was assayed and correlated to clinical outcome. It was demonstrated that SRL 172 was effective in inducing intracellular IL-2 responses in a significant number of patients with stage IV (AJCC) melanoma. Stanford et al. International Journal of Pharmaceutical Medicine 1999; 13: 191-195 report that there is increasing evidence that effective anti-tumour immune responses are likely to be mediated by type 1 cytokines. Recent investigations indicate that heat-killed Mycobacterium vaccae, is a reliable Th1 adjuvant and preliminary clinical trials indicate beneficial effects in melanoma, and cancer of the prostate and lung. More extensive controlled studies are currently being conducted to confirm these findings.

An unregulated or inappropriately regulated cellular immune response has also been observed during infection and particularly chronic infection, for example during progressive tuberculosis, lepromatous leprosy, visceral leishmaniasis and HIV infection and during allergies. By way of example, Clerici & Shearer G M Immunol Today 1993; 14: 107-11 propose that a Th1 to Th2 switch is a critical step in the etiology of HIV infection. Clerici & Shearer Immunol Lett 1996; 51: 69-73 show that HIV-specific cell mediated immunity may be the main correlater of protection against HIV infection and against the progression of HIV infection to AIDS. Abbot N C et al. European Journal of Vascular and Endovascular Surgery 2002 24:202-8 evaluated immunotherapy as a means of improving peripheral blood flow in chronic leprosy patients by administration of heat-killed Mycobacterium vaccae. It was shown that immunotherapy, given 18 months earlier, significantly improved blood flow and temperature sensation, in fully-treated, chronic, leprosy patients.

Accordingly, an aim of the present invention is to promote and establish the regulation of a cellular immune response, including the regulation or modulation of Th1 and/or Th2, in such a way so as to overcome the negative effects of the unregulated or inappropriately regulated cellular immune response.

Suitably, a composition according to the present invention may modulate the Th1 or Th2 response, i.e. a Th1 or Th2 response in a recipient that results in, for example, tissue damage.

Suitably, a composition according to the present invention may decrease the Th1 response and decrease the Th2 response. By way of example, such a composition may be useful in the prevention and/or treatment, preferably prevention, of diabetes for example. Suitably, a composition according to the present invention may increase the Th1 response without affecting the Th2 response. By way of example, such a composition may be useful as an immune enhancer.

Suitably, a composition according to the present invention may increase the Th1 response and decrease the Th2 response. By way of example, such a composition may be useful in the prevention or treatment (preferably prevention) of asthma.

Suitably, a skilled person can test a specific species of each genus according to the present invention to determine its specific Th1/Th2 response.

An unregulated or inappropriately regulated immune response may play a role in the establishment of disease due to the fact that some diseases cause shifted Th1 and/or Th2 responses. Accompanying these atypical Th1 and Th2 reactions are a series of abnormal inflammatory responses, which may take part in the mechanisms underlying tissue pathology.

By way of example only, a composition according to the present invention may counteract the disadvantages of reduced contact with environmental influences (for example, antigens) commensurate with modern life, may counteract the influence of treatment of an infection (for example a bacterial infection, a viral infection, such as HIV PMWS and PDNS, an infection caused by papilloma viruses, including equine sarcoid, genital warts and dysplasia of the uterine cervix that precedes carcinoma of the cervix, or a parasitic infection, such as malaria, and visceral infections); an immunological abnormality accompanying an infection; an autoimmune disease (e.g. a vascular disorder, such as obliterative vascular disorder, and the immunological aspects underlying myointimal hyperplasia and/or atheroma formation (otherwise known as arteriosclerosis), diabetes, arthritis and graft rejection); stress (for example, major trauma stress, psychosocial stress and chronic stress); an allergy (for example asthma including allergic asthma, hayfever, allergic dermatitis (eczema), allergies to plant contact or ingestion, stings—such as nettle and insect stings, and allergies to insect bites—such as midges, for instance Culicoides (which causes Sweet Itch in horses) and fleas); heaves; COPD; autism; SIPH (such as EIPH); cancer (for example leukaemia, rare solid tumours of childhood, melanoma, carcinoma, sarcoma or adenocarcinoma); an immune system imbalance (e.g. an immune system imbalance in children); and post-operative stress and infection in a recipient

### Vaccine

In the art, it is known that DNA vaccines, which are essentially DNA sequences attached to gold particles and which are fired into the skin by a helium gun, are efficient vaccine delivery systems. Unlike conventional vaccines, these DNA vaccines do not require a traditional adjuvant component. In accordance with a further aspect of the present invention, the immune modulator composition as defined herein may suitably be used in conjunction with such DNA vaccines to augment or participate in the influencing of the immune response.

The preparation of vaccines which contain one or more substances as an active ingredient(s), is known to one skilled in the art. Typically, such vaccines are prepared as injectables, either as liquid solutions or suspensions; solid forms suitable for solution in, or suspension in, liquid prior to injection may also be prepared. The preparation may also be emulsified, or the active ingredient(s) encapsulated in liposomes. The active ingredients are often mixed with excipients which are pharmaceutically acceptable and compatible with the active ingredient. Suitable excipients are, for example, water, saline, dextrose, glycerol, ethanol, or the like and combinations thereof. Alternatively, the vaccine may be prepared, for example, to be orally ingested and/or capable of inhalation.

In addition, if desired, the vaccine may contain minor amounts of auxiliary substances such as wetting or emulsifying agents and pH buffering agents.

A “preventative” or “prophylactic” vaccine is a vaccine which is administered to naive individuals to prevent development of a condition, such as by stimulating protective immunity.

A “therapeutic” vaccine is a vaccine which is administered to individuals with an existing condition to reduce or minimise the condition or to abrogate the immunopathological consequences of the condition.

Identifying a Bacterium that Modulates a Cellular Immune Response in an Infant Through Administration of the Bacterium to a Subject

In another aspect, the present invention relates to a method for identifying one or more whole cells of bacteria from a genus of aerobic organisms in the order of Actinomycetales that result in an immune response in a recipient following administration of a composition comprising same to a subject and exposing the recipient to a bodily fluid of the subject whilst the recipient is an infant, comprising the steps of: (a) contacting a first test subject with an immunostimulant and exposing an infant recipient to a bodily fluid of the test subject; (b) contacting a second test subject with an immunostimulant mixed with a bacterium and exposing an infant recipient to a bodily fluid of the test subject; (c) measuring the cellular immune response in each of the test infant recipients; and (d) comparing the cellular immune response in each of the test recipient infants, wherein, a lower cellular immune response in a recipient infant from exposure to a bodily fluid of a subject contacted with the immunostimulant mixed with a bacterium in comparison to the immunostimulant alone is indicative of a modification of the cellular immune response in an infant recipient resultant by the administration of the bacterium to the subject.

In another aspect, the present invention relates to a method of determining the Th1/Th2 response in an infant recipient following administration of a composition comprising a species of bacteria from a genus of aerobic organisms in the order of Actinomycetales to a subject and exposing the infant recipient to a bodily fluid of the subject comprising the steps of: which method comprises utilisation of the tuberculin skin test. In mice, the tuberculin skin test is preferably carried out on the foot pad. In a predominant Th1 reaction the positive foot pad immune response is maximal at 24 hours and diminishes at 48 hours. However, as the Th2 reactivity increases then the 48 hour positive foot pad immune response increases and can even exceed the foot pad immune response at 24 hour.

The effect of BCG vaccination is well documented using this tuberculin skin test. Thus, the test assay can be used to assess whether or not the introduction of an immune modulator composition to an infant recipient according to the present invention modulates the BCG cellular immune response.

As used herein, the term “test animal” refers to any infant that elicits a cellular immune response to the immunostimulant. Preferably, the test animal(s) is a mammal. More preferably, the test animal(s) is a rat, hamster, rabbit, guinea pig or mouse. More preferably, the test animal(s) is a mouse.

Preferably, a composition of the present invention modifies the T helper cell response in an infant Suitably, the composition of may modify the T helper cell response by decreasing the Th1 and Th2 response. Suitably, the composition may modify the T helper cell response by increasing the Th1 response and decreasing the Th2 response. Suitably, the composition may modify the T helper cell response by increasing the Th1 response without affecting the Th2 response.

Preferably, the immunostimulant will have a known Th1 and Th2 response. For example, with the immunostimulant BCG the reaction is usually largest at 24 h when it is an indicator of the Th1 response; the reaction at 48 h is usually less and includes a Th2 contribution. It is known that BCG predominantly stimulates a Th1 response.

By use of such immunostimulants it may be possible to determine the Th1/Th2 response of a test bacterium and, thus, it may be possible to identify one or more bacteria which give rise to a bodily fluid which have a desired Th1/Th2 response in a recipient to treat and/or prevent (preferably prevent) a particular disease and/or disorder.

Preferably, the cellular immune response is measured using the tuberculin skin test Vaccination with an immunostimulant—such as BCG—induces a response to skin-testing with tuberculin (a soluble preparation of Tubercle bacilli), when tested later. The local reaction is measured at various intervals, for example, 24 hours, 48 hours and 72 hours after injection of tuberculin. Briefly, an immunostimulant (e.g. BCG) is used that induces a positive immune response to tuberculin. In the test animal, the tuberculin skin test is preferably carried out on the foot pad. In a predominant Th1 reaction the positive foot pad immune response is usually maximal at 24 hours and diminishes at 48 hours. However, as the Th2 reactivity increases then the 48 hour positive foot pad immune response increases and can even exceed the foot pad immune response at 24 hour. Thus, the assay can be used to assess whether or not the introduction of an immune modulator composition according to the present invention modulates the cellular immune response.

Preferably, the immunostimulant is BCG.

The invention will now be further described by way of Examples, which are meant to serve to assist one of ordinary skill in the art in carrying out the invention and are not intended in any way to limit the scope of the invention.

### EXAMPLES

### Example 1

### Immunisation of Rats with Gordona and Subsequent Protection of Offspring from T. cruzi

### Protocol

Female rats were immunised with BE-G101 (0.1 mg of bacterial mass of Gordona bronchialis in 0.1 ml, i.e. 1 in 10 dilution of the 10 mg/ml stock suspensions), or placebo one week before mating with sires and a week after that.

The strain of Gordona bronchialis used in the Experiments is NCTC 10667 obtained from the National Collection of Type Culture (Health Protection agency, 61 Colindale Avenue, London, NW9 5HT). This strain was publicly available.

Half of the mothers were infected with T. cruzi, one week following mating (the time when they received the second injection of the immunotherapeutic agents), at different sites. A final injection of BE-G101 was given one week later.

Offspring were infected by subcutaneous injection of 10⁶ T. cruzi at weaning aged 21 days.

T. cruzi parasitemias were measured at 8 and 15 days post-infection

### Results

TABLE 1
PARASITEMAS (number of parasites/50
fields) AT DAY 8 POST-INFECTION

| | | | Median [range or 25-75 percentile] | | | | |
|---|---|---|---|---|---|---|---|
| Groups | n | means ± sd | Min | 25% | Median | 75% | Max |
| BE-G101 | 10 | 14.7 ± 14.9 | 1 | 2 | 7.5 | 26 | 40 |
| BE-G101 + | 6 | 10 ± 4 | 3 | 9 | 10.5 | 12 | 15 |
| T. cruzi | | | | | | | |
| Control | 8 | 32.1 ± 16.3 | 15 | 21.5 | 27 | 40 | 65 |

Overall difference p<0.0005
BE-G101 vs control p<0.05

TABLE 2
PARASITEMIAS(number of parasites/50 fields) AT DAY 15

| | | | Median [range or 25-75 percentile] | | | | |
|---|---|---|---|---|---|---|---|
| Groups | n | means ± sd | Min | 25% | Median | 75% | Max |
| BE-G101 | 10 | 5.1 ± 5.4 | 0 | 1 | 3 | 9 | 15 |
| BE-G101 + | 6 | 8 ± 12.5 | 1 | 2 | 2 | 8 | 33 |
| T. cruzi | | | | | | | |
| Control | 8 | 15.4 ± 7.6 | 2 | 11 | 15.5 | 21.5 | 25 |

Overall difference p<0.001
BE-G101 vs control p<0.008

Statistical analysis was made by non-parametric tests because parasitemias deviate from a normal distribution. Tests employed were Kruskall-Wallis analysis of variance followed by the Mann-Whitney U test.

As can be seen, administration of whole cells of Gordona bronchialis to a subject resulted in protection of an infant against parasitemias on infection with T. cruzi. It is speculated that this may be because G. bronchialis behaves as an adjuvant and improves the immune response of the mother and thus improves the anti-T. cruzi response in the mother which is further transferred to the infant.

### Example 2

### Experiment to Demonstrate Transplacental and/or Oral Exposure of an Infant with a Bodily Fluid Component of a Rat Immunised with BE-G101 Results in Protection Against T. cruzi in the Infant

An experiment has been designed to test whether protection in an infant occurs via exposure to a bodily fluid component during gestation (e.g. transplacental exposure) and/or during feeding (e.g. oral exposure).

### Protocol

Female rats are immunised with BE-G101 (0.1 mg of bacterial mass of Gordona bronchialis in 0.1 ml, i.e. 1 in 10 dilution of the 10 mg/ml stock suspensions), or placebo one week before mating with sires and a week after that.

Half of the off-spring of the female rats immunised with BE-G101 are removed from their mother at birth and are weaned on the milk of the control female rats that had not been immunised with BE-101.

Likewise, half the off-spring of the control female rats are removed from their mother at birth and are weaned on the milk of the BE-G101 immunised rats.

Offspring were infected by subcutaneous injection of 10⁶ T. cruzi at weaning aged 21 days.

T. cruzi parasitemias were measured at 8 and 15 days post-infection

It is expected that the off-spring of the BE-G101 rats that are weaned on a control rat will be protected from T. cruzi infection suggesting that bodily fluid components capable of protecting an infant from T. cruzi are capable of crossing the placenta during gestation.

Furthermore, it is expected that the off-spring of the control rats that are weaned on the BE-G101 rats will also be protected from T. cruzi infection suggesting that bodily fluid components capable of protecting an infant from T. cruzi are present in the milk of BE-G101 immunised lactating rats.

All publications mentioned in the above specification are herein incorporated by reference. Various modifications and variations of the described methods and system of the present invention will be apparent to those skilled in the art without departing from the scope and spirit of the present invention. Although the present invention has been described on connection with specific preferred embodiments, it should be understood that the invention as claimed should not be unduly limited to such specific embodiments. Indeed, various modifications of the described modes for carrying out the invention which are obvious to those skilled in biochemistry. immunology and biotechnology or related fields are intended to be within the scope of the following claims.

1. Use of a composition comprising a whole cell of a bacterium from a genus of aerobic organisms in the order of Actinomycetales in the manufacture of a medicament for modulating the immune response of a recipient wherein said composition is administered to a subject and said recipient is exposed to a bodily fluid of said subject whilst said recipient is an infant.

2. Use of a composition according to claim 1 wherein the medicament is for the prevention and/or treatment of one or more of: an infection, an allergy, an autoimmune based disease and a neoplasm.

3. Use according to claim 1 wherein said bacterium is from one or more of the following genera: Gordonia, Rhodococcus, Dietzia, Nocardia, Tsukamurella, and Mycobacterium.

4. Use according to claim 3 wherein said bacterium is from the genus Gordonia.

5. Use according to claim 1 wherein the bacterium is G. bronchialis.

6. Use according to claim 1 wherein the subject is a mammal.

7. Use according to claim 1 wherein the medicament is for the prevention or treatment of an infection in said recipient.

8. Use according to claim 2 wherein the infection is by Trypanosoma cruzi.

9. Use according to claim 1 wherein the composition is administered to said subject prior to, during and/or after pregnancy of said subject.

10. Use according to claim 1 wherein the bodily fluid is milk or a component thereof.

11. Use according to claim 1 wherein the bodily fluid is blood or a component thereof.

12. Use according to claim 1 wherein the composition is administered to said subject prior to and/or during pregnancy with said recipient, such that the immunity of said recipient is modulated.

13. Use according to claim 12 wherein the infant is exposed to the bodily fluid of the subject via the placenta.

14. Use according to claim 1 wherein the composition is administered to said subject prior to lactation and the recipient is fed with the milk, such that the immunity of said recipient is modulated.

15. Use according to claim 1 wherein the protected immune response is enhanced.

16. Use according to claim 1 wherein the subject is the parent of the recipient.

17. A method of modulating the immune response of a recipient comprising administering an effective amount of a composition comprising whole cells of a bacterium from a genus of aerobic organisms in the order of Actinomycetales to a subject and exposing the recipient to a bodily fluid of said subject whilst said recipient is an infant.

18. A method according to claim 17 wherein the composition is administered to said subject prior to, during and/or after pregnancy of said subject.

19. A method according to claim 17 wherein the bodily fluid is milk or a component thereof.

20. A method according to claim 17 wherein the bodily fluid is blood or a component thereof.

21. A method according to claim 17 wherein the composition is administered to said subject prior to and/or during pregnancy with said infant, such that the immunity of said infant is modulated.

22. A method according to claim 21 wherein the infant is exposed to the bodily fluid of the subject via the placenta.

23. A method according to claim 17 wherein the composition is administered to said subject prior to lactation and feeding the infant with the milk, such that the immunity of said infant is modulated.

24. A method according to claim 17 wherein the composition is administered to prevent and/or treat one or more of the diseases or disorders selected from the group consisting of: an infection, an allergy, an autoimmune based disease and a neoplasm.

25. A method according to claim 17 wherein said bacterium is from one or more of the following genera: Gordonia, Rhodococcus, Nocardia, Dietzia, Tsukamurella, and Mycobacterium.

26. A method according to claim 25 wherein said bacterium is from the genus Gordonia.

27. A method according to claim 26 wherein the bacterium is G. bronchialis.

28. A method according to claim 17 wherein the subject is a mammal.

29. A method according to claim 17 wherein the composition is administered for the prevention or treatment of an infection in said recipient.

30. A method according to claim 29 wherein the infection is by Trypanasoma cruzi.

31. A method according to claim 17 wherein the immune response is enhanced.

32. A method according to claim 17 wherein the subject is the parent of the recipient.

33.-34. (canceled)
